(19) United States
(12) Patent Application Publication
(10) Pub. No.: US 2004/0043981 A1
(43) Pub. Date: Mar. 4, 2004

(54) Pharmaceutical composistions

(76) Inventors: Stefan Horkovics-Kovats, Angath (AU); Johannes Raneburger, Worgl (AU); Franz Xaver Schwarz, Worgl (AU)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/399,427

(22) PCT Filed: Oct. 18, 2001
(86) PCT No.: PCT/EP01/12076

(30) Foreign Application Priority Data
Oct. 20, 2000 (AT) A18062000

(51) Int. Cl. A61K 31/407; C07D 487/04

(52) U.S. Cl. 514/210.09; 540/350

(57) ABSTRACT

Clavulanic acid, in which the median of the particles is from 8 μm to 35 μm and their use in pharmaceutical compositions.

The present invention relates to pharmaceutical composition, such as pharmaceutical compositions of clavulanic acid in combination with amoxicillin.

AUGMENTIN®, see e.g. Merck Index 12th Edition, Item 2402, is a commercial form of pharmaceutical compositions of the β-lactamase inhibitor clavulanic acid (in the form of the potassium salt) in combination with the broad-spectrum antibiotic amoxicillin (in the form of the trihydrate). When orally administered, the active ingredient amoxicillin and the β-lactamase inhibitor of such compositions should be available in dissolved form at the resorption site in reasonable amounts and concentrations.

According to methods as conventional in the preparation of clavulanic acid, clavulanic acid and its salts may be obtained, e.g. in the form of needles, rod-shaped crystals or rosettes; in a grain size and grain size distribution such, that 80% of the particles, e.g. crystalline particles, have a grain size of 40 μm and more, e.g. up to 250 μm and more, and the median of the particles is of 70 μm and more, e.g. up to 110 μm and more. The grain size and distribution of grain size may be determined by known methods, e.g. by pattern analysis or laser diffraction processes. The median is a central value that can be used instead of an average value. In the case of grain size, the median means that the actual grain size of 50% of the particles is larger and of 50% of the particles is smaller than the median. The median may be determined by a method as conventional, e.g. by numeric calculation based on the actual grain size distribution.

We have now surprisingly found that clavulanic acid may be resorbed particularly well from the gastrointestinal tract following oral administration if the median, and/or the grain size, of the clavulanic acid particles, are of a certain size.

In one aspect, the present invention provides clavulanic acid, e.g. in the form of a potassium salt, in which

the median of the particles is between 8 μm, preferably 10 μm, and 35 μm, preferably 30 μm, and/or in which

80% of the particles of the clavulanic acid have a grain size of 1 μm to 70 μm, such as 2 μm to 70 μm.

The median is preferably from 10 μm to 30 μm, more preferably from 12 μm to 30 μm. Preferably 80% of the particles of the clavulanic acid have a grain size of 2 μm to 70 μm. Clavulanic acid includes clavulanic acid in free form and clavulanic acid in the form of a salt, e.g. a pharmaceutically acceptable salt, such as an alkali or alkaline earth salt, preferably a potassium salt. Clavulanic acid according to the present invention is in the form of solid particles, e.g. in powder or crystalline form.

It has surprisingly also been found that clavulanic acid particles according to the present invention keeps its favourable resorption characteristics, if administered in the form of pharmaceutical compositions, e.g. AUGMENTIN® compositions.

In another aspect, the present invention provides a pharmaceutical composition containing clavulanic acid, in which

the median of the clavulanic acid particles is from 8 μm, preferably from 10 μm, to 35 μm, preferably to 30 μm; and/or in which

80% of the clavulanic acid particles, e.g. crystalline particles or powder particles, of the clavulanic acid, have a grain size of 1 μm to 70 μm, such as 2 μm to 70 μm.

A pharmaceutical composition includes oral pharmaceutical compositions, such as tablets, film-coated tablets, chewing tablets, powders for oral suspensions and dispersible tablets.

Clavulanic acid according to the present invention may be obtained e.g. as follows:

Clavulanic acid, preferably in the form of a potassium salt, may be isolated, e.g. crystallised, from a solvent, preferably n-butanol and/or isobutanol, and the crystals obtained, e.g. still moist with solvent, are treated in a combined drier/mixer until the median of the particles is from 8 μm to 35 μm, and/or 80% of the clavulanic acid particles have a grain size of 1 μm to 70 μm.

Surprisingly we have found that, if n-butanol and/or isobutanol is used as a solvent in clavulanic acid (potassium salt) preparation, the clavulanic acid particles may be produced in a size which is particularly useful for obtaining clavulanic acid according to the present invention by following treatment in a combined drier and mixer compared with solvents other than n-butanol and/or isobutanol.

In another aspect, the present invention provides a process for the production of clavulanic acid in which the median of particles is between 8 μm and 35 μm, and/or in which 80% of the clavulanic acid particles have a grain size of 1 μm to 70 μm, comprising the steps

a) isolating clavulanic acid, e.g. in the form of a potassium salt, from organic solvent, e.g. n-butanol and/or isobutanol, and

b) treating the particles obtained in step (a) in a combined drier/mixer until the median of the particles is between 8 μm and 35 μm, and/or until 80% of the clavulanic acid particles have a grain size of 1 μm to 70 μm.

Isolation of clavulanic acid in the form of a pharmaceutically acceptable salt from n-butanol and/or isobutanol is known and is described e.g. in WO 97/18216. The content of WO 97/18216 and the content of the literature cited therein are incorporated in the present application by reference as a part of the present application.

In WO 97/18216, a process is described, according to which clavulanic acid in n-butanol and/or isobutanol as the solvent is converted into a pharmaceutically acceptable salt of clavulanic acid, e.g. an alkali or alkaline earth salt, preferably a potassium salt. As a starting material, clavulanic acid may be used as such or in the form of a salt, e.g. a lithium salt or an amine salt, preferably an amine salt. Amine salts include salts of clavulanic acid with an amine as disclosed in WO 97/18216, e.g. tert.-butylamine, tert.-octylamine (2-amino-2,4, 4-trimethylpentane), N,N′-diisopropylethylenediamine, N,N,N′, N′-tetramethyl-diaminoethane and 1,3-bis(dimethylamino)-2-propanol, preferably tert.-octylamine or tert.butylamine.

Clavulanic acid in the form of a salt with an amine may be produced e.g. according to one of the methods disclosed in WO 97/18216 including the literature cited therein, preferably as follows:

a) Fermentating an appropriate micro-organism, e.g. a micro-organism which is capable of producing clavulanic acid during fermentation, to obtain an impure aqueous fermentation broth containing clavulanic acid;

b) optionally harvesting a part of the fermentation broth obtained in step a),

c) optionally pre-purifying a fermentation broth of step a) or b), e.g. by

removing at least part of the solids from the fermentation broth, and/or

extracting an impure or pre-purified aqueous fermentation broth containing clavulanic acid, with an organic solvent which is able to form two phases when in contact with water;

to obtain a pre-purified aqueous fermentation broth or aqueous solution containing clavulanic acid;

d) optionally concentrating an impure or pre-purified fermentation broth or aqueous solution of step a), b) or c);

e) acidifying a fermentation broth of step a), b), c) or d) to obtain an acidified impure or pre-purified, optionally concentrated, aqueous fermentation broth or solution containing clavulanic acid;

f) extracting an acidified fermentation broth or solution of step e) with an organic solvent in which clavulanic acid is soluble under the conditions of extraction, and which is able to form two phases when in contact with water, to obtain a solution of clavulanic acid in an organic solvent;

g) treating a solution obtained in step f) with an amine, preferably tert.-butylamine, tert.-octylamine, N,N′-diisopropylethylenediamine, N,N,N′,N′-tetramethyl-diaminoethane or 1,3-bis(dimethylamino)-2-propanol, more preferably tert.-octylamine or tert.-butylamine, to obtain clavulanic acid in the form of a salt with an amine, and/or in the form of a solvate, such as an acetone solvent; and optionally isolating the salt obtained; and

h) converting a salt of step g) into a pharmaceutically acceptable salt of clavulanic acid, e.g. a potassium salt.

Conversion according to step h) may be carried out as appropriate, e.g. as follows:

Clavulanic acid in the form of a salt with an amine is dissolved in n-butanol and/or isobutanol. It is preferable to use either n-butanol or isobutanol, e.g. in an amount that is sufficient to dissolve the clavulanic acid. Water, e.g. 0.5 to 10%, e.g. 1.0 to 5%, such as 1.0 to 4%, e.g. 1.5 to 3.0%, may be present in the solution. The solution obtained is optionally treated with activated carbon, and is brought into contact with a source of cation which is capable of forming a pharmaceutically acceptable salt with clavulanic acid. Cation sources of this kind are described in WO 97/18216, for example in literature cited therein, and include e.g. alkaline earth or alkali salts of a (C₂₋₈)-carboxylic acid, e.g. 2-ethylhexanoic acid, for example the potassium salt thereof, as well as acetates, e.g. potassium acetate. If an acetate is used as a source of cation, acetic acid may be additionally added to the reaction mixture. Contact of the cation source with the solution of an amine salt of clavulanic acid may take place according to one of the methods disclosed in WO 97/18216, for example in literature cited therein, and is preferably effected as follows:

A solution of the cation source in a solvent, preferably in n-butanol and/or isobutanol, is added, e.g. in portions, to a solution of an amine salt of clavulanic acid. At least one equivalent of the cation source, preferably 1.0, such as about 1.1, to preferably 3.0, such as about 2.0 equivalents, are used per mol of clavulanic acid (salt). Clavulanic acid in the form of a pharmaceutically acceptable salt may precipitate from the reaction mixture. For example, in order to complete precipitation, a further solvent in which the pharmaceutically acceptable salt is poorly soluble may be added to the mixture, and/or the mixture obtained may be cooled e.g. to temperatures of below 0° C. to about 10° C., such as about 0° C. to about 5° C. The pharmaceutically acceptable salt of clavulanic acid is isolated, e.g. by filtration, centrifugation, and is obtained in solid form, e.g. in crystalline form, either rosette-free or in the form of rosettes.

The pharmaceutically acceptable salt of clavulanic acid, e.g. whilst moist with solvent, is dried in a drier which is combined with a mixer, whereby the shearing force of the mixer is set such that clavulanic acid is obtained, in which the median of the particles is from 8 μm to 35 μm and/or 80% of the particles, e.g. crystal or powder particles, of the clavulanic acid have a grain size of 1 μm to 70 μm. Driers combined with a mixer are commercially available. The shearing force of a mixer and thus the particle size of the clavulanic acid may be regulated by switching on the mixer for a shorter or longer period during drying. The most suitable lengths of time that the mixer should be switched on and off may be determined by preliminary tests.

Clavulanic acid according to the present invention, in the form of a pharmaceutically acceptable salt in which the median of the particles is from 8 μm to 35 μm, and/or in which 80% of the particles, e.g. crystal or powder particles, of the clavulanic acid have a grain size of 1 μm to 70 μm, may be obtained.

In another aspect, the present invention provides a process for the preparation of clavulanic acid in the form of a pharmaceutically acceptable salt, such as a potassium salt, in which the median of the particles is from 8 μm to 35 μm and/or in which 80% of the clavulanic acid particles have a grain size of 1 μm to 70 μm, said process comprising the steps

a) converting an amine salt of clavulanic acid into a pharmaceutically acceptable salt of clavulanic acid, e.g. wherein

said amine salt is obtained by treating a solution of clavulanic acid in an organic solvent with an amine, e.g. wherein

said solution of clavulanic acid in an organic solvent is obtained by extracting an acidified aqueous, impure or pre-purified fermentation broth or solution containing clavulanic acid with an organic solvent in which clavulanic acid is soluble under the conditions of extraction, and which is able to form two phases when in contact with water, e.g. wherein

said acidified impure or pre-purified fermentation broth or solution containing clavulanic acid is obtained by acidifying an optionally pre-concentrated aqueous, impure or pre-purified fermentation broth or solution containing clavulanic acid, e.g.

wherein

said optionally pre-concentrated aqueous, impure or pre-purified fermentation broth or solution containing clavulanic acid is obtained by optionally concentrating an impure or pre-purified fermentation broth or aqueous solution containing clavulanic acid, e.g. wherein

said pre-purified aqueous fermentation broth or aqueous solution containing clavulanic acid is obtained by removing at least part of the solids from a fermentation broth containing calvulanic acid, and/or by extracting an impure or pre-purified aqueous fermentation broth containing clavulanic acid, with an organic solvent which is able to form two phases when in contact with water; e.g.

wherein

said impure aqueous fermentation broth containing clavulanic acid is obtained by fermentating an appropriate micro-organism, and

b) treating the pharmaceutically acceptable salt of clavulanic acid obtained in a), e.g. in a form which is moist with solvent, in a combined drier and mixer until the median of the particles is from 8 μm to 35 μm and/or until 80% of the particles have a grain size of 1 μm to 70 μm, e.g. the shearing force of the mixer is used to adjust the appropriate median and/or grain size, e.g. by appropriate switching the mixer on and off during drying.

An aqueous solution of clavulanic acid may also be produced, e.g. by dissolving clavulanic acid in an aqueous solvent system.

In the following examples, all temperatures are in degree Centigrade and are uncorrected.

The following abbreviations are used:

CS—K: Clavulanic acid in the form of a potassium salt

### EXAMPLE A

CS—K is produced according to a method of examples 1 to 9 of WO 97/18216 and is isolated from the reaction mixture. Crystalline particles having a median greater than 35 μm and wherein less than 80% of the particles have a grain size between 1 μm and 70 μm are obtained. CS—K thus obtained is treated whilst moist with solvent, according to any of the following examples:

### EXAMPLE 1

CS—K obtained according to example A, in which the median of the particles is 103 μm and in which 80% of the particles have a grain size of 40 μm to 250 μm (hereinafter referred to as “CS-B”), in a form which is moist with solvent, is treated in a drier in which the particles can be simultaneously mixed and dried. The mixer is switched on and off during drying, so that CS—K particles are obtained in which the median of the particles is 25 μm and in which 80% of the particles have a grain size of 5 μm to 60 μm (hereinafter referred to as “CS-A”). The number of times to switch the mixer on and off and the mixing time to obtain CS-A is determined in a preliminary test.

### EXAMPLE 2

CS—K obtained according to example A, in which the median of the particles is 87 μm and in which 80% of the particles have a grain size of 50 μm to 200 μm (hereinafter referred to as “CS—D”) is treated as described in example 1 but using different mixing times and mixing switches. CS—K particles are obtained in which the median of the particles is 18 μm and in which 80% of the particles have a grain size of 2 μm to 50 μm (hereinafter referred to as “CS—C”).

### EXAMPLE 3

Film-Coated Tablets Amoxicillin in the form of a trihydrate and cross-linked starch are mixed, granulated with water, dried and equalised. The granulate obtained is mixed with CS-A or CS-B, cellulose, talcum and magnesium stearate, and the mixture obtained is pressed into tablets. Tablets “A” or “B” are obtained, which contain per tablet 1.0 g of amoxicillin in the form of a trihydrate (corresponds to 0.875 g of amoxicillin), cross-linked starch, cellulose, talcum and magnesium stearate and

in tablet “A” 0.149 g of “CS-A” (corresponds to 0.125 g of clavulanic acid), and

in tablet “B” 0.149 g of “CS-B” (corresponds to 0.125 g of clavulanic acid).

Tablets “A” and “B” are coated with a film composition. Film-coated tablets “A” or film-coated tablets “B” are obtained, which comprise of tablets “A” or “B”, and which are coated per tablet with a film coating comprising of film-forming components, pigments, plasticisers.

### EXAMPLE 4

Chewing Tablets

Amoxicillin in the form of a trihydrate, sugar alcohols and colourant are mixed, the mixture obtained is granulated with water, dried and equalised. The granulate obtained is mixed with CS—C or CS—D, sweeteners, aromatizers, cross-linked starch, talcum and magnesium stearate, and the mixture obtained is pressed into tablets. Chewing tablets “C” or “D” are obtained, which contain per tablet 0.466 g of amoxicillin in the form of a trihydrate (corresponds to 0.4 g of amoxicillin), sugar alcohols, sweeteners, aromatic substances, cross-linked starch, talcum and magnesium stearate and

in Chewing tablet “C” 0.068 g of CS—C (corresponds to 0.057 g of clavulanic acid); and

in Chewing tablet “D” 0.068 g of CS—D (corresponds to 0.057 g of clavulanic acid).

### EXAMPLE 5

Powders for oral suspensions Amoxicillin in the form of a trihydrate, CS—C or CS—D, carboxylic acids, sweeteners, aromatizers, thickeners, sugar alcohols and silicon dioxide are admixed. Powders for oral suspensions “C” or “D” are thereby obtained. 23 g portions of the obtained powders for oral suspensions “C” or “D” are filled into glass bottles (multi-dose containers). Prior to administration, 84 ml of water is added to this bottle. 100 ml of a ready-to-use, oral suspension “C” or “D” is obtained, representing 20 doses each of 5 ml. Each dose contains 0.466 g of amoxicillin in the form of a trihydrate (corresponding to 0.4 g of amoxicillin), carboxylic acids, sweeteners, aromatizers, thickeners, sugar alcohols, silicon dioxide and

in oral suspension “C” 0.068 g of CS—C (corresponds to 0.057 g of clavulanic acid);

in oral suspension “D” 0.068 g of CS—D (corresponds to 0.057 g of clavulanic acid).

Testing for Bioavailability

To 24 healthy probands (test persons) are administered

a film-coated tablet “A” or a film-coated tablet “B”

a chewing tablet “C” or a chewing tablet “D”

an oral suspension “C” or an oral suspension “D”.

The bioavailability of clavulanic acid in the administered medicinal forms is determined in accordance with the guidelines of the European Agency for the Evaluation of Medicinal Products (EMEA), Human Medicines Evaluation Unit, Draft of the Committee for proprietary Medicinal Products (CPMP) under the title “Note for guidance on the investigation of bioavailability and bioequivalence” of Dec. 17, 1998 (CPMP/EWP/QWP/1401/98), in keeping with the relevant EU and ICH Guidelines and Regulations.

Cₘₐₓ is the maximum plasma concentration.

AUC is the area under the plasma concentration curve, extrapolated to t=infinite.

The AUC (μg×h/ml) and Cₘₐₓ (μg/ml) values indicated in TABLE 1 below are obtained for each respective pharmaceutical composition used:

TABLE 1

| Medicinal form | AUC (μg × h/ml) | Cₘₐₓ (μg/ml) |
|---|---|---|
| film-coated tablets “A” | 6.6 +/− 2.3 | 3.1 +/− 1.1 |
| film-coated tablets “B” | 6.0 +/− 2.3 | 2.7 +/− 1.1 |
| chewing tablets “C” | 3.0 +/− 0.85 | 1.45 +/− 0.4 |
| chewing tablets “D” | 2.6 +/− 0.83 | 1.3 +/− 0.4 |
| oral suspension “C” | 3.2 +/− 1.0 | 1.6 +/− 0.4 |
| oral suspension “D” | 2.8 +/− 1.1 | 1.4 +/− 0.5 |

From TABLE 1, it is immediately evident that the bioavailability of clavulanic acid in film-coated tablets “A”, chewing tablets “C” and oral suspensions “C”, which contain CS—K particles having a median=25 μm, 80% grain size of 5 μm to 60 μm, or a median=18 μm, 80% grain size of 2 μm to 50 μm, respectively, is improved, for at least 10%, compared with film-coated tablets “B”, chewing tablets “D” and oral suspensions “D”, which contain CS—K particles having a median=103 μm and 80% grain size of 40 μm to 250 μm, or a median=87 μm and 80% grain size of 50 μm to 200 μm, respectively.

1. Clavulanic acid or a salt thereof in which the median of the particles is from 8 μm to 35 μm.

2. Clavulanic acid or a salt thereof according to claim 1, wherein 80% of the particles of the clavulanic acid have a grain size of 1 μm to 70 μm.

3. Clavulanic acid or a salt thereof, in which
80% of the particles of the clavulanic acid have a grain size in the range of 2 μm to 70 μm and
the median of the particles is between 10 μm and 30 μm.

4. Clavulanic acid according to any one of claims 1 to 3, wherein the clavulanic acid is in the form of a potassium salt.

5. A pharmaceutical composition comprising clavulanic acid or a salt thereof as defined in any one of claims 1 to 4.

6. A pharmaceutical composition according to claim 5, which is a tablet, film-coated tablet, chewing tablet, powder for oral suspensions, or dispersible tablet.

7. A process for the production of clavulanic acid as defined in any one of claims 1 to 4, comprising the steps of
a) isolating clavulanic acid from an organic solvent, and
b) treating the particles obtained in step a) in a combined drier/mixer until the median of the particles is between 8 μm and 35 μm.

8. A process according to claim 7, wherein in step a) the organic solvent is n-butanol and/or isobutanol.

9. A process for the production of clavulanic acid or a salt thereof as defined in any one of claims 1 to 4, comprising
a) fermentating an appropriate micro-organism to obtain an impure aqueous fermentation broth containing clavulanic acid;
b) optionally harvesting a part of the fermentation broth obtained in step a),
c) optionally pre-purifying a fermentation broth of step a) or b), to obtain a pre-purified aqueous fermentation broth or aqueous solution containing clavulanic acid;
d) optionally concentrating an impure or pre-purified fermentation broth or aqueous solution of step a), b) or c);
e) acidifying a fermentation broth of step a), b), c) or d) to obtain an acidified impure or pre-purified, optionally concentrated, aqueous fermentation broth or solution containing clavulanic acid;
f) extracting an acidified fermentation broth or solution of step e) with an organic solvent in which clavulanic acid is soluble under the conditions of extraction, and which is able to form two phases when in contact with water, to obtain a solution of clavulanic acid in an organic solvent;
g) treating the solution obtained in step f) with an amine, to obtain clavulanic acid in the form of a salt with an amine,
h) converting a salt of step g) into a pharmaceutically acceptable salt of clavulanic acid, and
i) treating the particles obtained in step h) in a combined drier/mixer until the median of the particles is between 8 μm and 35 μm.
